(19) United States
(12) Patent Application Publication
(10) Pub. No.: US 2024/0376520 A1
(43) Pub. Date: Nov. 14, 2024

(54) METHODS FOR FRAGMENTING COMPLEMENTARY DNA

(71) Applicants: The Regents of the University of California, Oakland, CA (US); CZ Biohub SF, LLC, San Francisco, CA (US)

(72) Inventors: Derek Bogdanoff, Berkeley, CA (US); Chang Kim, Berkeley, CA (US); Tomasz Nowakowski, Berkeley, CA (US)

(21) Appl. No.: 18/563,307

(22) PCT Filed: May 26, 2022
(86) PCT No.: PCT/US22/31130

§ 371 Date: Nov. 21, 2023

Related U.S. Application Data
(60) Provisional application No. 63/195,510, filed on Jun. 1, 2021.

(51) Int. Cl. C12Q 1/6806; C12N 15/10

(52) U.S. Cl. CPC C12Q 1/6806; C12N 15/1093

(57) ABSTRACT

The present disclosure provides a method for fragmenting cDNA on a support, where such fragmenting allows for application of short-read sequencing to longer cDNAs. In this method, the fragments of a cDNA molecule each receive the same unique molecular identifier (UMI), thereby allowing assembly of cDNA sequences from multiple short read sequences. Thus, the sequencing, transcript reconstruction and transcript analysis may be done at single molecule resolution.

### CROSS-REFERENCE

This application claims the benefit of U.S. Provisional Patent Application No. 63/195,510, filed Jun. 1, 2021, which application is incorporated herein by reference in its entirety.

### INCORPORATION BY REFERENCE OF SEQUENCE LISTING PROVIDED AS A TEXT FILE

A Sequence Listing is provided herewith as a text file, “CZBH-003WO_SEQ_LIST_ST25.txt” created on Apr. 25, 2022 and having a size of 2 KB. The contents of the text file are incorporated by reference herein in their entirety.

### INTRODUCTION

The detection of nucleic acids having specific nucleotide sequences present in a biological sample has been used, for example, as a method for identifying and classifying microorganisms, diagnosing infectious diseases, detecting and characterizing genetic abnormalities, identifying genetic changes associated with cancer, studying genetic susceptibility to disease, and measuring response to various types of treatment. A common technique for detecting specific nucleic acid sequences in a biological sample is nucleic acid sequencing. Furthermore, the availability of large-scale parallel nucleic acid sequencing allows for identification of sequence variation within complex populations and detection of rare sequence variations.

### SUMMARY

The present disclosure provides a method for fragmenting cDNA on a support, where such fragmenting allows for application of short-read sequencing to longer cDNAs. In this method, the fragments of a cDNA molecule each receive the same unique molecular identifier (UMI), thereby allowing assembly of cDNA sequences from multiple short read sequences. Thus, the sequencing, transcript reconstruction and transcript analysis may be done at single molecule resolution. This, in turn, allows one to distinguish between different isoforms.

### BRIEF DESCRIPTION OF THE DRAWINGS

FIG. 1A-IC describe an exemplary workflow for reconstructing transcript sequences using the present method.

FIG. 2 schematically illustrates some of the steps summarized in FIG. 1A, in more detail.

FIG. 3 schematically illustrates some of the cDNA produced made using some embodiments of the present method.

### DEFINITIONS

The terms “molecular index,” “molecular identifier sequence,” “identifier sequence,” and “tag sequence that identifies” are used interchangeably herein to refer to a sequence of nucleotides used to identify and/or track the source of a polynucleotide. After the polynucleotides in a sample are sequenced, the identifier sequence can be used to distinguish the sequence reads and/or determine from which sample a sequence read is derived. An “identifier sequence” may be referred to a “sample barcode”, “index” or “indexer” sequence. For example, different samples (e.g., polynucleotides derived from different individuals, different tissues or cells, or polynucleotides isolated at different times points), can be tagged with identifier sequences that are different from one another. After sequencing, the source of a polynucleotide (e.g., a cDNA fragment) can be tracked back to a particular sample using the molecular index. Identifier sequences can be added to a sample by ligation, by primer extension using a tailed primer that contains an identifier sequence in a 5′ tail, or using a transposon. A molecular index can range in length from 2 to 100 nucleotide bases or more and may include multiple subunits, where each different identifier has a distinct identity and/or order of subunits. A sample identifier sequence may be added to the 5′ end of a polynucleotide or the 3′ end of a polynucleotide, for example. In some cases, a molecular index has a length in range of from 1 to 36 nucleotides, e.g., from 6 to 30 nucleotides, or 8 to 20 nucleotides. In certain cases, the molecular index may be error-correcting, meaning that even if there is an error (e.g., if the sequence of the molecular barcode is mis-synthesized, mis-read or is distorted by virtue of the various processing steps leading up to the determination of the molecular barcode sequence) then the code can still be interpreted correctly. Descriptions of exemplary error correcting sequences can be found throughout the literature (e.g., US20100323348 and US20090105959, which are both incorporated herein by reference). In some cases, a molecular index may be of relatively low complexity (e.g., may be composed of a mixture of 8 to 1024 different sequences), although higher complexity identifier sequences can be used in some cases.

The term “sample identifier sequence” is a sequence of nucleotides that is appended to a target polynucleotide, where the sequence identifies the sample (e.g., which individual, which cell, which tissue, or which times points, etc.) from which a sequence read is derived. In use, each sample is tagged with a different sample identifier sequence (e.g., one sequence is appended to each sample, where the different samples are appended to different sequences), and the tagged samples can be pooled. After the samples are sequenced, the sample identifier sequence can be used to identify the source of the sequences.

As used herein the term “adaptor” refers to a nucleic acid that can comprise one or more of a barcode, a primer binding sequence, a capture sequence, a sequence complementary to a capture sequence, unique molecular identifier (UMI) sequence, an affinity moiety, and a restriction site.

A “transposome” comprises an integration enzyme such as an integrase or a transposase, and a nucleic acid comprising an integration recognition site, such as a transposase recognition site. In embodiments provided herein, the transposase can form a functional complex with a transposase recognition site that is capable of catalyzing a transposition reaction. The transposase may bind to the transposase recognition site and insert the transposase recognition site into a target nucleic acid in a process sometimes termed “tagmentation”. In some such insertion events, one strand of the transposase recognition site may be transferred into the target nucleic acid. In one example, a transposome comprises a dimeric transposase comprising two subunits, and two non-contiguous transposon sequences. In another example, a transposome comprises a transposase comprises a dimeric transposase comprising two subunits, and a contiguous transposon sequence.

Generally, a “barcode” is a nucleic acid that includes one or more nucleotide sequences that can be used to identify one or more particular nucleic acids. The barcode can be an artificial sequence, or can be a naturally occurring sequence generated during transposition, such as identical flanking genomic DNA sequences (g-codes) at the end of formerly juxtaposed DNA fragments. In some cases, a barcode is an artificial sequence that is absent in (not normally present in) the target nucleic acid sequence and can be used to identify one or more target nucleic acids. A barcode can comprise at least about 1, 2, 3, 4, 5, 6, 7, 8, 9, 10, 11, 12, 13, 14, 15, 16, 17, 18, 19, 20 or more consecutive nucleotides. In some cases, a barcode comprises at least about 10, 20, 30, 40, 50, 60, 70 80, 90, 100 or more consecutive nucleotides. In some cases, at least a portion of the barcodes in a population of nucleic acids comprising barcodes is different. In some cases, at least about 10%, 20%, 30%, 40%, 50%, 60%, 70%, 80%, 90%, 95%, 99% of the barcodes are different. In other instances, all of the barcodes are different. The diversity of different barcodes in a population of nucleic acids comprising barcodes can be randomly generated or non-randomly generated.

There are generally three types of identifier sequence used herein. The first type is a cell identifier sequence, which identifies the cell from which a cDNA is made. In this case, the cell identifier sequence differs from the cell to cell and can be used to assign sequence to a particular cell. The second type is a support identifier sequence, which differs from support to support (e.g., bead to bead). This identifier sequence can be used to assign sequence to a particular support (e.g., a particular bead). The third type of identifier is a molecular identifier, which differs from molecule to molecule. This identifier sequence can be used to assign different sequence reads as being derived from the same molecule.

A “sequencing adaptor” or “sequencing adaptor site” refers to a region of a nucleic acid that comprises one or more sites that can hybridize to a primer. In some cases, a nucleic acid can include at least a first primer site useful for amplification, sequencing, and the like. Exemplary sequences of sequence binding sites include, but are not limited to AATGATACGGCGACCACCGAGATCTACAC (SEQ ID NO:1) (P5 sequence) and CAAGCAGAAGACGGCATACGAGAT (SEQ ID NO:2) (P7 sequence).

As used herein, the term “sequencing read” and/or grammatical equivalents thereof can refer to a repetitive process of physical or chemical steps that is carried out to obtain signals indicative of the order of monomers in a polymer. The signals can be indicative of an order of monomers at single monomer resolution or lower resolution. In some instances, the steps can be initiated on a nucleic acid target and carried out to obtain signals indicative of the order of bases in the nucleic acid target. The process can be carried out to its typical completion, which is usually defined by the point at which signals from the process can no longer distinguish bases of the target with a reasonable level of certainty. If desired, completion can occur earlier, for example, once a desired amount of sequence information has been obtained. A sequencing read can be carried out on a single target nucleic acid molecule or simultaneously on a population of target nucleic acid molecules having the same sequence, or simultaneously on a population of target nucleic acids having different sequences. In some cases, a sequencing read is terminated when signals are no longer obtained from one or more target nucleic acid molecules from which signal acquisition was initiated. For example, a sequencing read can be initiated for one or more target nucleic acid molecules that are present on a solid phase substrate and terminated upon removal of the one or more target nucleic acid molecules from the substrate. Sequencing can be terminated by otherwise ceasing detection of the target nucleic acids that were present on the substrate when the sequencing run was initiated. Exemplary methods of sequencing are described in U.S. Pat. No. 9,029,103, which is incorporated herein by reference in its entirety

The terms “next-generation sequencing” or “high-throughput sequencing”, as used herein, refer to the so-called parallelized sequencing-by-synthesis or sequencing-by-ligation platforms currently employed by Illumina, Life Technologies, and Roche, etc. Next-generation sequencing methods may also include nanopore sequencing methods such as those commercialized by Oxford Nanopore Technologies, electronic-detection based methods such as Ion Torrent technology commercialized by Life Technologies, or single-molecule fluorescence-based methods commercialized by Pacific Biosciences.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a cDNA molecule” includes a plurality of such cDNA molecules and reference to “the transposome” includes reference to one or more transposomes and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides a method for fragmenting cDNA on a support. The fragmenting allows for application of short-read sequencing to longer cDNAs. In general, full length cDNA (e.g., cDNA longer than about 500 bases) is synthesized from target RNA, where the cDNA includes at least one molecular index (e.g., a barcode identifying the origin of the target RNA). In a first step, the cDNA undergoes a tagmentation reaction on transposomes tethered to a solid support, generating first cDNA fragments that are tethered to the support and that comprise a “support barcode” (a barcode tethered to the support). In a second step, the tethered first cDNA fragments are contacted with a plurality of untethered transposomes, where the untethered transposomes comprise a transposase and a nucleic acid comprising a polymerase chain reaction (PCR) primer amplification sequence; the contacting of the second step results in tagmentation of the first cDNA fragments to generate second cDNA fragments that include: i) the support barcode; and ii) a PCR primer amplification sequence at both ends.

Thus, in some cases, a method of the present disclosure for fragmenting double-stranded cDNA on a support comprises: a) contacting double-stranded cDNA (“input cDNA”) with a plurality of transposomes that are tethered to a support, wherein the tethered transposomes comprise: (i) a transposase; and (ii) a nucleic acid that is tethered to the support and comprises a support barcode and a PCR primer amplification sequence, where the contacting step (a) results in tagmentation of the cDNA to produce first cDNA fragments that are tethered to the support; and b) contacting the product of step (a) with a plurality of untethered transposomes, wherein the untethered transposomes comprise: (i) a transposase; and ii) a nucleic acid that comprises a PCR primer amplification sequence; wherein the contacting step (b) results in tagmentation of at least some of the first cDNA fragments of (a) to produce second cDNA fragments that have: i) the support barcode and ii) a PCR primer amplification sequence at both ends. In some cases, wherein the nucleic acid of step (a)(ii) comprises i. transposon ends that are bound to the transposase, ii. the support barcode, iii the PCR primer amplification sequence and iv. an end that is tethered to the support. In some cases, the nucleic acid of (b)(ii) comprises i. transposon ends that are bound to the transposase and ii. the PCR primer amplification sequence. In some cases, after step (a), the transposase present in the tethered transposome is removed, e.g., by contacting the tethered transposome with sodium dodecyl sulfate. In some cases, the transposase present in the untethered transposome is removed.

Generally, the support to which the tethered transposome is tethered is a solid support. Exemplary solid supports include but are not limited to nanoparticles, beads, hydrogel molecules, flow cell surfaces, and column matrices. In some cases, the solid support is a bead. In some cases, the method comprises use of a plurality of transposomes, each of which is tethered to (immobilized on) a different solid support, such that the method comprises use of a plurality of solid supports. In some cases, the plurality of solid supports is plurality of beads.

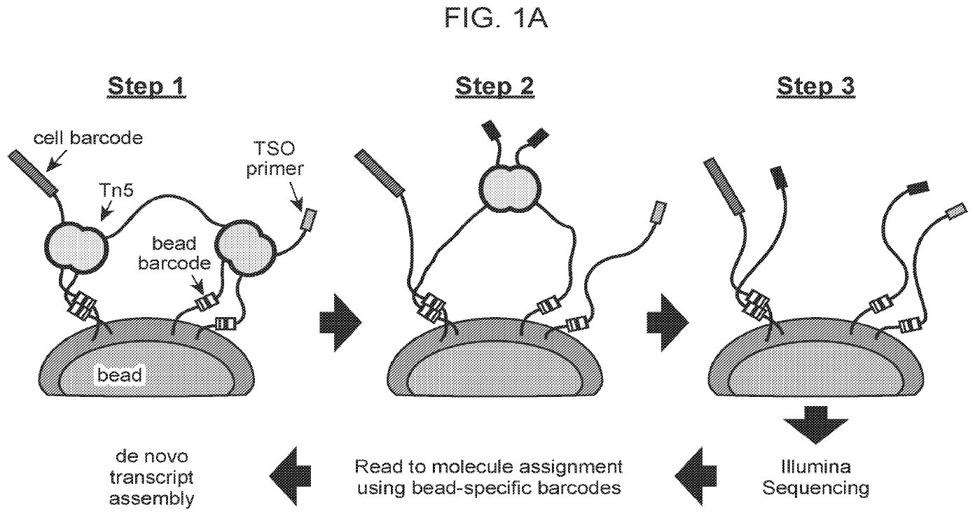

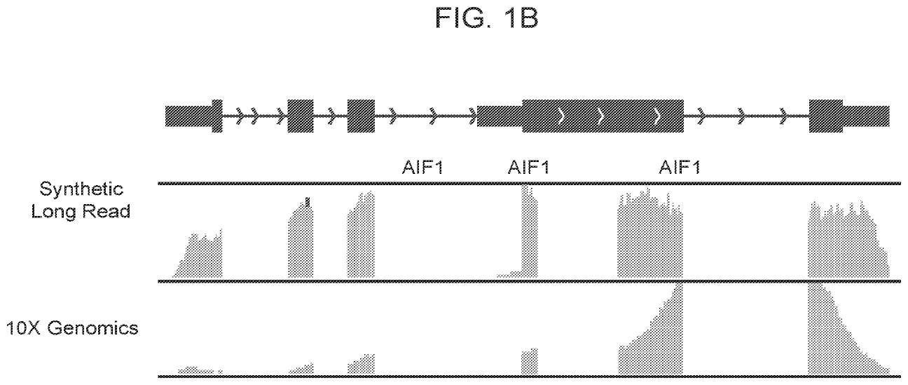

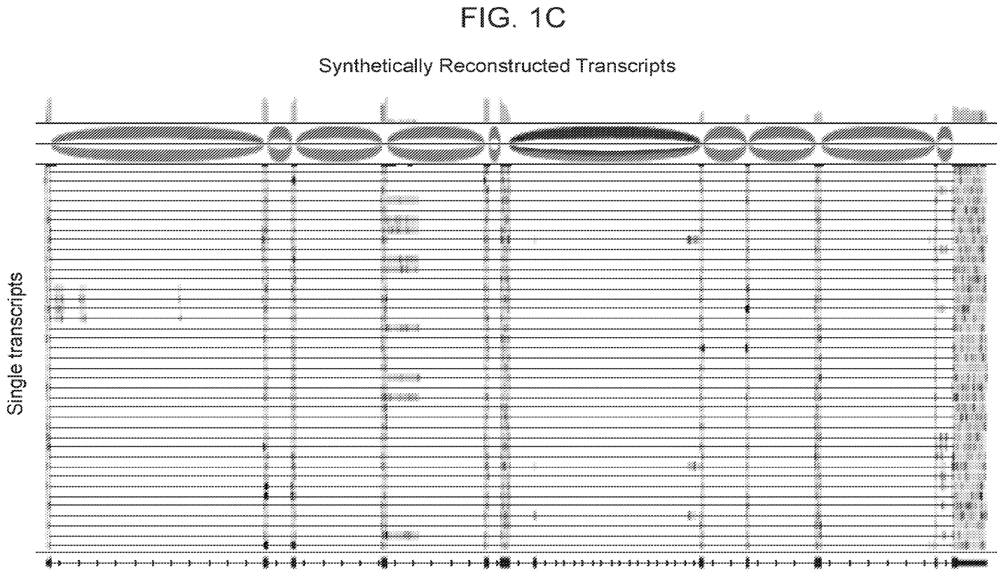

FIG. 1A-C shows an exemplary workflow for reconstructing cDNA sequences.

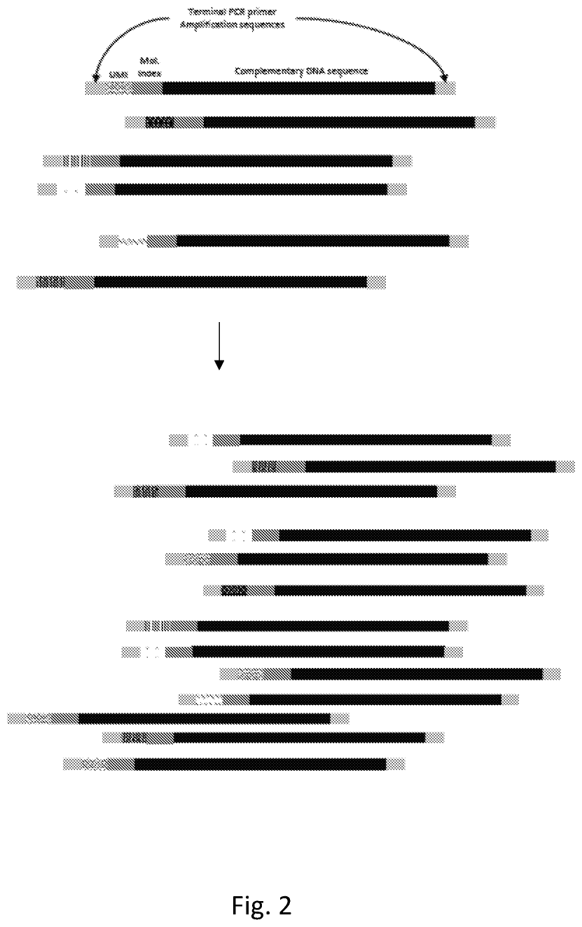

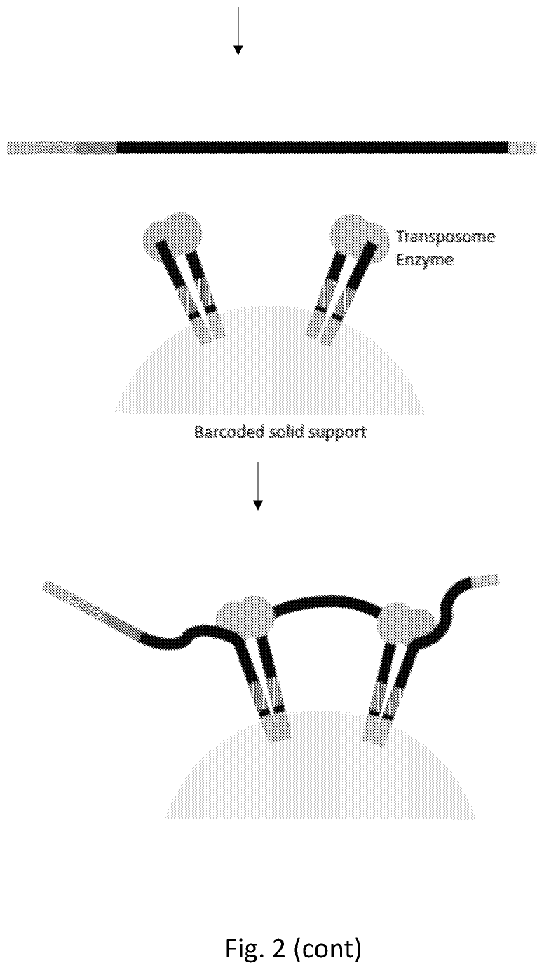

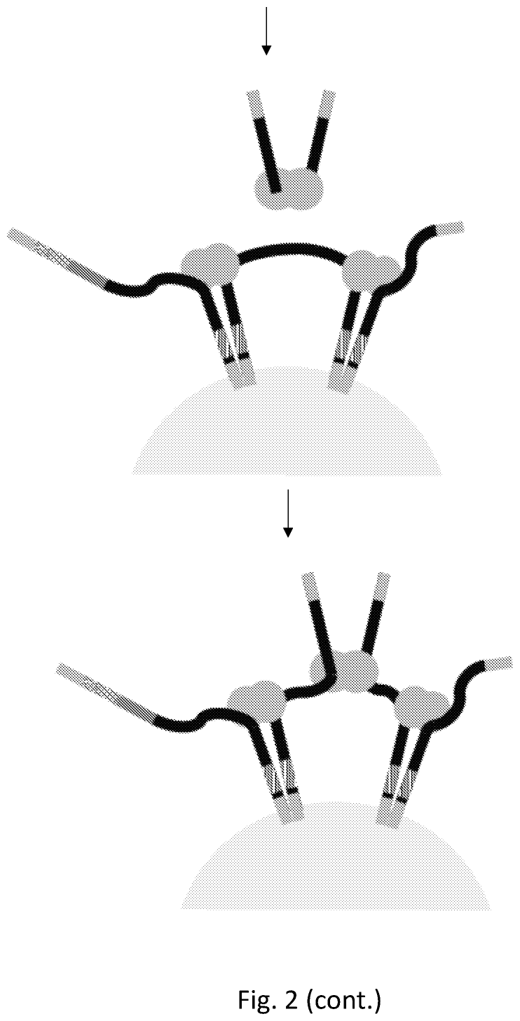

FIG. 2 illustrates one implementation of the method. FIG. 2 shows full length cDNA molecules containing terminal PCR primer amplification sequences, molecular indices (cell or spatial barcode) and UMIs. These molecules are amplified by PCR, then tagmented on a barcoded solid support. Secondary cDNA tagmentation is mediated by an untethered transposome, the transposase is removed by SDS. The fragmented template is amplified by PCR to create a short-read library.

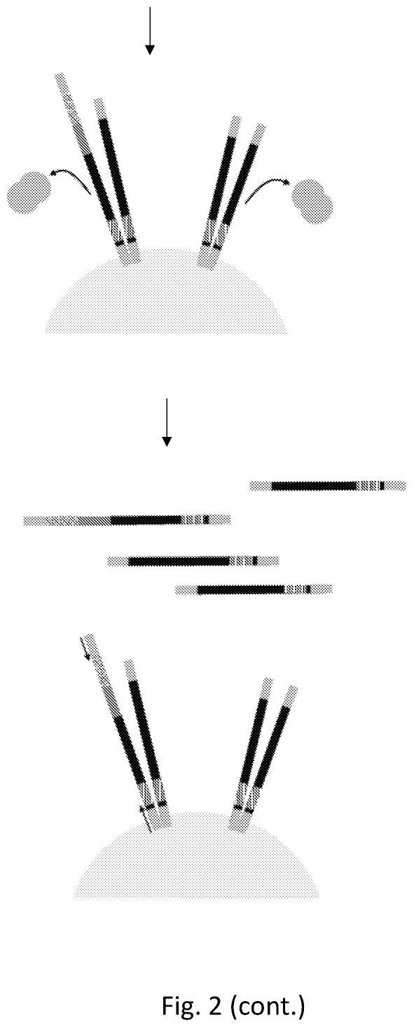

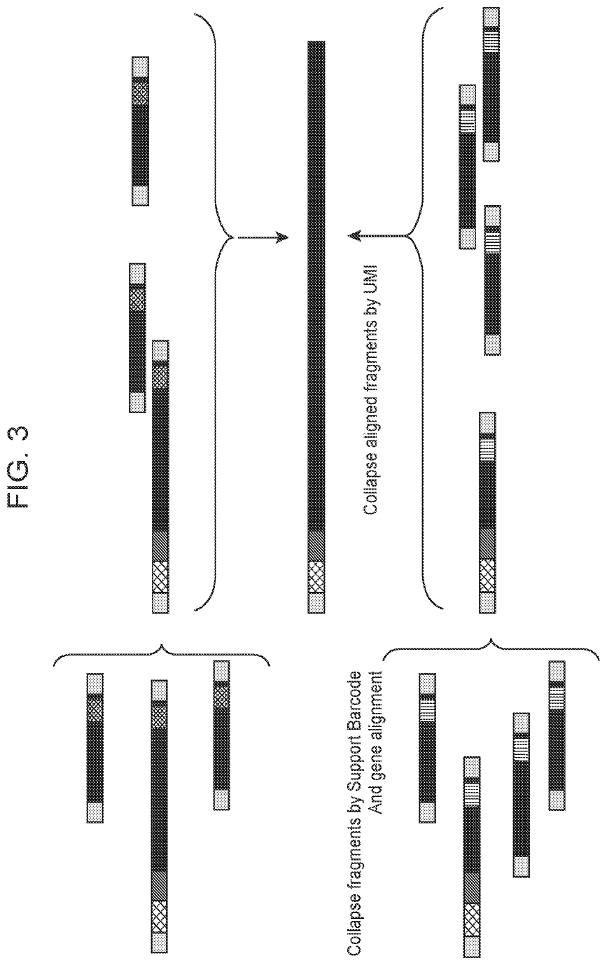

FIG. 3 shows an example of how cDNA sequences can be reconstructed on a molecule-by-molecule basis using the UMI, support (bead) identifier.

Certain details of this method may be described below.

The product of the method, second cDNA fragments, include: a) a cDNA fragment corresponding to a 3′ terminal portion of the input cDNA, where the cDNA fragment comprises: i) a molecular index (e.g., a cell/spatial barcode); ii) a UMI; iii) a support barcode (e.g., a bead barcode); and iv) PCR primer amplification sequences; b) cDNA fragments corresponding to internal portions of the input cDNA where the cDNA fragment includes: i) a support barcode (e.g., a bead barcode); and ii) PCR primer amplification sequences; and c) a cDNA fragment corresponding to a 5′ terminal portion of the input cDNA, where the cDNA fragment comprises: i) a support barcode (e.g., a bead barcode); ii) a PCR primer amplification sequence; and iii) a template switching oligonucleotide (TSO) sequence (“TSO amplification handle”).

In some cases, the method further comprises amplifying the first cDNA fragments and the second cDNA fragments to produce first cDNA amplification products and second cDNA amplification products, respectively. In some cases, the method further comprises amplifying the second cDNA fragments to produce second cDNA amplification products. In some cases, the method further comprises sequencing the amplification products to produce sequence reads. Once the cDNA fragments are sequenced, the support bar codes can be used to identify amplification products that originated from the same single RNA molecule or cell. In other words, the support bar codes provide contiguity information. Those cDNA fragments that are tagged with a particular support barcode (having a particular nucleotide sequence) can be assumed to originate from a single input cDNA.

In some cases, the input cDNA comprises a polymerase chain reaction (PCR) primer amplification sequence at one or both ends of the input cDNA. In some cases, the input cDNA comprises a molecular index at one or both ends. For example, the molecular index is a nucleic acid barcode that provides information regarding the origin of the target RNA that served as the template for generating the input cDNA. The molecular index can provide information regarding the cell type, the cell status (e.g., diseased; non-diseased), the fluid type, the organ type, the organ status, and the like.

A method of the present disclosure provides for determining the sequence of a long cDNA using a short-read sequencing method (e.g., Illumina), where the short-read sequence method is applied to the cDNA fragments generated by the method, and the contiguity information provided by the support barcode is used to link the sequences of the cDNA fragments to provide the nucleotide sequence of the input cDNA.

In some cases, the input cDNA contains a molecular index sequence which can be read by short-read sequencing on one or more of the sequenced cDNA fragments. In some cases, information from the input cDNA molecular index read on one cDNA fragment can be shared with fragments that do not contain the molecular index, but which do originate from the same input cDNA molecule by way of a support barcode. In some cases, additional sequence information can be used to delineate molecular origin of a sequenced cDNA fragment (e.g., sequence alignment position to a reference sequence), when a support barcode is not sufficient to do so alone, (e.g., when multiple input cDNA molecules share the same support barcode).

An input cDNA can have a length of more than 500 nucleotides, more than 700 nucleotides, more than 1,000 nucleotides, more than 5,000 nucleotides, more than 10,000 nucleotides, up to about 20,000 nucleotides. In some cases, an input cDNA has a length of from about 500 nucleotides to 20,000 nucleotides; e.g., in some cases, an input cDNA has a length of from about 500 nucleotides to about 1,000 nucleotides, from about 1,000 nucleotides to about 5,000 nucleotides, from about 5,000 nucleotides to about 10,000 nucleotides, from about 10,000 nucleotides to about 15,000 nucleotides, or from about 15,000 nucleotides to about 20,000 nucleotides. The cDNA fragments generated using a method of the present disclosure, and that can then be sequenced (e.g., using a short-read sequencing method) are generally no longer than about 700 nucleotides. In some cases, cDNA fragments generated using a subject method are from about 100 nucleotides to about 700 nucleotides in length; e.g., in some cases, cDNA fragments generated using a subject method are from about 100 nucleotides to about 200 nucleotides, from about 200 nucleotides to about 300 nucleotides, from about 300 nucleotides to about 400 nucleotides, from about 400 nucleotides to about 500 nucleotides, from about 500 nucleotides to about 600 nucleotides, or from about 600 nucleotides to about 700 nucleotides in length.

### Supports

As used herein, some embodiments of the method may use barcoded particles, e.g., beads. In these embodiments, the particles are coated in oligonucleotides, where the surface-tethered oligonucleotides on each particle have a unique sequence that is different to the sequence that is in the oligonucleotides that are tethered to other particles in the population. In other words, if there are 1,000 barcoded particles, the oligonucleotides that are tethered to each particle will have a unique sequence. This identifier for one particle is different to the identifier for other particles.

Such barcoded particles may be made by emulsion PCR, which method has been successfully used for other applications and is described in, e.g., Kanagal-Shamanna et al (Methods Mol Biol 2016 1392: 33-42) and Shao et al (PlosOne 2011 0024910). In some embodiments, the method may involves coating a population of particles with a forward primer (e.g., via click chemistry, streptavidin, or via a covalent interaction), combining the particles with a reverse primer, dNTPs, polymerase and an oligonucleotide template that has a 5′ sequence that hybridizes with the forward primer, a variable, e.g., random, sequence that produces the UMIs when copied and a 3′ sequence corresponding to the reverse primer, producing an emulsion, where each droplet contains on average a single particle, a single molecule of template, and multiple molecules of reverse primers, and thermocycling the emulsion, thereby grafting copies of the sequence of the template onto the forward primers. Some aspects of emulsion PCR are described by Dressman et al. (PNAS 2003 100: 8817-8822). As would be understood, the template molecules may have a forward primer binding site, a degenerate (e.g., random) sequence of 6-10 nucleotides (or even more random nucleotides dependent on the number of unique particles required) and sequence that provides a binding site for the reverse primer, when it is copied. Other sequences may be in the template.

### Target RNA

The input cDNA is a copy of a target RNA. Where a plurality of input cDNAs are used in a subject method, the plurality of input cDNAs are copies of target RNAs. Target RNA includes RNA from any source, including prokaryotic cells, archaeal cells, and eukaryotic cells. Target RNA includes ribosomal RNA (rRNA), transfer RNA (tRNA), micro RNA (miRNA), messenger RNA (mRNA).

Sources of target RNA include, but are not limited to, organelles, cells, tissues, bodily fluids, organs, and whole organisms. Cells that may be used as sources of target nucleic acid molecules may be prokaryotic (bacterial cells, for example, Escherichia, Bacillus, Serratia, Salmonella, Staphylococcus, Streptococcus, Clostridium, Chlamydia, Neisseria, Treponema, Mycoplasma, Borrelia, Legionella, Pseudomonas, Mycobacterium, Helicobacter, Erwinia, Agrobacterium, Rhizobium, and Streptomyces genera); archeaon, such as crenarchaeota, nanoarchaeota or euryarchaeotia; or eukaryotic such as fungi, (for example, yeasts), plants, protozoans and other parasites, and non-mammalian animals (including insects, arthropods, marine animals, etc.), and mammals (for example, rodents (rat, mouse), ungulates (e.g., bovines, equines, ovines, etc.), canines, felines, non-human primates. and humans).

A target RNA may be obtained from a biological sample or a patient sample (e.g., sample from a human patient). The term “biological sample” or “patient sample” as used herein includes samples such as tissues and bodily fluids. “Bodily fluids” may include, but are not limited to, blood, serum, plasma, saliva, cerebral spinal fluid, pleural fluid, tears, lactal duct fluid, lymph, sputum, urine, amniotic fluid, and semen. A sample may include a bodily fluid that is “acellular.” An “acellular bodily fluid” includes less than about 1% (w/w) whole cellular material. Plasma or serum are examples of acellular bodily fluids. A sample may include a specimen of natural or synthetic origin (i.e., a cellular sample made to be acellular).

In some cases a target RNA may be derived from an environmental or metagenomic sample, wherein the species, or plurality of species, where a plurality of species includes organisms of different genetic composition, from which the material is derived is unknown.

In some cases, a target RNA is from a normal (non-diseased) mammalian cell. In some cases, a target RNA is from a diseased mammalian cell. Diseased cells include, e.g., cancer cells.

In some cases, a target RNA is a sequence variant. A “sequence variant” or “sequence variation” can be any variation with respect to a reference sequence. A sequence variation may consist of a change in, insertion of, or deletion of a single nucleotide, or of a plurality of nucleotides (e.g. 2, 3, 4, 5, 6, 7, 8, 9, 10, or more nucleotides). Where a sequence variant comprises two or more nucleotide differences, the nucleotides that are different may be contiguous with one another, or discontinuous. Non-limiting examples of types of sequence variants include single nucleotide polymorphisms (SNP), deletion/insertion polymorphisms (DIP), copy number variants (CNV), short tandem repeats (STR), simple sequence repeats (SSR), variable number of tandem repeats (VNTR), amplified fragment length polymorphisms (AFLP), retrotransposon-based insertion polymorphisms, sequence specific amplified polymorphism, and differences in epigenetic marks that can be detected as sequence variants (e.g. methylation differences). In some cases, a sequence variant is a haplotype.

In some cases, the RNAs are all from a single cell. In some cases, the RNAs are a plurality of RNAs from a single cell. In some cases, the RNAs are a subset of all RNAs in a single cell. For example, Genomics single-cell Gel Bead-In Emulsions (GEMS) can be used to partition cells into single cells. In some cases, the RNAs are from multiple cells, tissues, fluids, organs, or whole organisms.

A cDNA copy/ies of a target RNA(s) are generated using any known method. Suitable methods include, e.g., emulsion based single-cell RNA sequencing (scRNA SEQ), array-based spatial transcriptomics, and the like. See, e.g., Moncada et al. (2020) Nature Biotechnol. 38:333; Chen et al. (2016) BMC Genomics 17 Supp. 7:508; Slovin et al. (2021) Methods Mol. Biol. 2284:343; and Marx (2021) Nature Methods 18:9. In some cases, full-length cDNA is generated using the target RNA.

In some cases, cDNA is generated from a plurality of RNAs, where a plurality of RNAs includes RNAs that differ in nucleotide sequence from one another. A plurality of RNAs can include from 10 RNAs to 10⁹ different RNAs (RNAs that differ in nucleotide sequence from one another). For example, a plurality of RNAs can include from 10 RNAs to 10² RNAs, from 10² RNAs to 10³ RNAs, from 10³ RNAs to 10⁴ RNAs, from 10⁴ RNAs to 10⁵ RNAs, from 10⁵ RNAs to 10⁶ RNAs, from 10⁶ RNAs to 10⁷ RNAs, from 10⁷ RNAs to 10⁸ RNAs, or from 10⁸ RNAs to 10⁹ RNAs. A plurality of RNAs can include from 2 to 10 RNAs, or from 10 to 10² RNAs.

In some cases, cDNA is generated from a single species of RNA, where a species constitutes and identical nucleotide sequence. In some cases, a single species of RNA can be targeted for cDNA generation. In some cases, a plurality of single species of RNA can be targeted for cDNA generation.

### Transposases

Any known transposase is suitable for use herein. In some cases, the transposase is a wild-type transposase. In some cases, the transposase is a mutant transposase that comprises one or more mutations relative to a wild-type transposase, where the mutant transposase retains the ability to catalyze cleavage of a cDNA and addition of an adaptor nucleic acid (e.g., a nucleic acid comprising one or more of a barcode sequence, a PCR primer amplification sequence, etc.) to the 3′- and 5′-cleavage ends (where the 3′ cleavage end of the cDNA may be referred to as a “proximal” end and where the 5′ cleavage end of the cDNA may be referred to as a “distal” end). Suitable transposases include: a prokaryotic transposase, a Tn transposase, a MuA transposase, a Vibhar transposase, HERMES, Ac-Ds, Ascot-1, Bs1, Cin4, Copia, En/Spm, F element, hobo, Hsmar1, Hsmar2, IN (HIV), IS1, IS2, IS3, IS4, IS5, IS6, IS10, IS21, IS30, IS50, IS51, IS150, IS256, IS407, IS427, IS630, IS903, IS911, IS982, IS1031, ISL2, L1, Mariner, P element, Tam3, Tc1, Tc3, Tel, THE-1, Tn/O, TnA, Tn3, Tn5, Tn7, Tn10, Tn552, Tn903, Tol1, Tol2, Tnl0, and Ty1. In some cases, the transposase is a Tn5 transposase, e.g., a wild-type Tn5 transposase or a variant of a wild-type Tn5 transposase.

Non-limiting examples of suitable transposases include: a hyperactive Tn5 transposase and a Tn5-type transposase recognition site (Goryshin and Reznikoff, J. Biol. Chem., 273:7367 (1998)), or MuA transposase and a Mu transposase recognition site comprising R1 and R2 end sequences (Mizuuchi, K., Cell, 35: 785, 1983; Savilahti, H, et al., EMBO J., 14: 4893, 1995). An exemplary transposase recognition site that forms a complex with a hyperactive Tn5 transposase (e.g., EZ-Tn5T″ Transposase, Epicentre Biotechnologies, Madison, Wis.) comprises the following 19b transferred strand (sometimes “M” or “ME”) and non-transferred strands: 5′ AGATGTGTATAAGAGACAG 3′ (SEQ ID NO:3), 5′ CTGTCT CTTATACACATCT 3′ (SEQ ID NO:4), respectively. ME sequences can also be used as optimized by a skilled artisan. More examples of transposition systems that are suitable for use include Staphylococcus aureus Tn552 (Colegio et al., J. Bacteriol., 183: 2384-8, 2001; Kirby et al., Mol. Microbiol., 43: 173-86, 2002), Ty1 (Devine & Boeke, Nucleic Acids Res., 22: 3765-72, 1994; and WO 95/23875), Transposon Tn7 (Craig Science. 271: 1512, 1996; Craig, Review in: Curr Top Microbiol Immunol., 204:27-48, 1996), Tn/O and IS10 (Kleckner et al., Curr Top Microbiol Immunol., 204:49-82, 1996), Mariner transposase (Lampe et al., EMBO J., 15: 5470-9, 1996), Tc1 (Plasterk, Curr. Topics Microbiol. Immunol., 204: 125-43, 1996), P Element (Gloor, Methods Mol. Biol., 260: 97-114, 2004), Tn3 (Ichikawa & Ohtsubo, J Biol. Chem. 265:18829-32, 1990), bacterial insertion sequences (Ohtsubo & Sekine, Curr. Top. Microbiol. Immunol. 204: 1-26, 1996), retroviruses (Brown et al., Proc Natl Acad Sci USA, 86:2525-9, 1989), and retrotransposon of yeast (Boeke & Corces, Annu Rev Microbiol. 43:403-34, 1989). Additional examples include IS5, Tn10, Tn903, IS911, Sleeping Beauty, SPIN, hAT, PiggyBac, Hermes, TcBuster, AeBuster1, Tol2, and engineered versions of transposase family enzymes (Zhang et al., (2009) PLoS Genet. 5:e1000689. Epub 2009 Oct. 16; Wilson C. et al (2007) J. Microbiol. Methods 71:332-5).

### Utility

As noted above, a method of the present disclosure provides for determining the sequence of a long cDNA using a short-read sequencing method (e.g., Illumina), where the short-read sequence method is applied to the cDNA fragments generated by the method, and the contiguity information provided by the support barcode is used to link the sequences of the cDNA fragments to provide the nucleotide sequence of the input cDNA.

A method of the present disclosure can be used in a wide variety of research, therapeutic, and diagnostic applications. For example, differential RNA isoform usage has been shown to be essential in delineating cellular identity and function, and has been demonstrated as an accurate biomarker in a wide ranges of diseases. RNA isoform identification and measurement is applicable to, e.g., molecular research in fields such as oncology and organismal development; and also to medical diagnostics. Identification and quantification of RNA isoforms present in a sample is of interest because different RNA isoforms can later be translated as different proteins. Detection of RNA isoforms whose presence or quantity varies between samples may lead to new biomarkers and highlight novel biological processes that cannot be detected at the genomic level. A method of the present disclosure can also be used in transcriptome profiling by RNA sequencing; see, e.g., Di et al. (2019) Proc. Natl. Acad. Sci. USA 117:2886.

In some cases, a method of the present disclosure is used to detect differential RNA isoform usage and/or missplicing, e.g., in diseased vs. non-diseased states, in development, and in other cellular processes. See, e.g., Polymenidou et al. (2011) Nat. Neurosci. 14:459; and Bernard et al. (2014) Bioinformatics 30:2447

In some cases, a subject method is useful for detecting a sequence variant. In some cases, detecting a sequence variant comprises detecting mutations (e.g. rare somatic mutations) with respect to a reference sequence or in a background of no mutations, where the sequence variant is correlated with disease. In general, sequence variants for which there is statistical, biological, and/or functional evidence of association with a disease or trait are referred to as “causal genetic variants.” A single causal genetic variant can be associated with more than one disease or trait. In some embodiments, a causal genetic variant can be associated with a Mendelian trait, a non-Mendelian trait, or both. Causal genetic variants can manifest as variations in a polynucleotide, such 1, 2, 3, 4, 5, 6, 7, 8, 9, 10, 20, 50, or more sequence differences (such as between a polynucleotide comprising the causal genetic variant and a polynucleotide lacking the causal genetic variant at the same relative genomic position). Non-limiting examples of types of causal genetic variants include single nucleotide polymorphisms (SNP), deletion/insertion polymorphisms (DIP), copy number variants (CNV), short tandem repeats (STR), restriction fragment length polymorphisms (RFLP), simple sequence repeats (SSR), variable number of tandem repeats (VNTR), randomly amplified polymorphic DNA (RAPD), amplified fragment length polymorphisms (AFLP), inter-retrotransposon amplified polymorphisms (IRAP), long and short interspersed elements (LINE/SINE), long tandem repeats (LTR), mobile elements, retrotransposon microsatellite amplified polymorphisms, retrotransposon-based insertion polymorphisms, sequence specific amplified polymorphism, and heritable epigenetic modification (for example, DNA methylation). A causal genetic variant may also be a set of closely related causal genetic variants. Some causal genetic variants may exert influence as nucleotide sequence variations in RNA molecules. At this level, some causal genetic variants are also indicated by the presence or absence of a species of RNA polynucleotides.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example

### Synthetic Long Read Library Preparation

Barcoded cDNA underwent bead based tagmentation by the addition of 10 ng of cDNA to 1 μL of bead based transposome and 11 μL of 2×TAPS-DMF buffer (20 mM TAPS-NaOH (pH 8.5), 10 mM MgCl2, 20% DMF), followed by incubation at 37° C. for 30 minutes with agitation. After the initial tagmentation reaction, 1 μL of P5 adapter containing Tn5 was added, and the reaction was incubated in the same conditions for an additional 15 minutes. Beads were then washed twice in 1×B&W buffer containing 0.1% sodium dodecyl sulfate (SDS) to release the bound Tn5. Sequencing libraries were amplified off the bead bound templates by sequential PCR reactions utilizing combinations of TruSeq P5 and TruSeq P7 primers for amplification of the 3′ cDNA barcode/UMI containing fragments, Nextera P5 and TruSeq P7 for internally derived fragments, and template switching oligonucleotide (TSO) sequence and TruSeq P7 for the 5′ terminal cDNA sequence. All PCR reactions were run by resuspending the beads in 1×NEBNext High-Fidelity 2×PCR Master Mix (NEB) with the addition of each primer at 1 M. After each PCR reaction, the bead bound template was collected by incubation on a magnet for further reactions or storage at 4° C., and the library containing supernatant was cleaned using a 0.78×SPRI bead cleanup. Short read sequencing libraries were quantified and quality controlled by Agilent High Sensitivity Bioanalyzer assay, prior to sequencing on an Illumina NovaSeq 6000 sequencer using the read lengths 48×8×8×98.

### Synthetic Long Read Reconstruction at Single Molecule Resolution

Reads were mapped to the transcriptome and demultiplexed with their cell, molecular, and transposase barcode. Transposase barcodes were used to link the 3′ cell and molecular barcodes to the internal reads. Aligned reads were then grouped together by their mapped gene, and matching cell and molecular barcode. These groups of paired-end reads were bridged across to reconstruct the full reads based on the overlapping reads with different transposase barcodes. The reconstructed reads were then remapped to the most likely transcript and quantified to create a cell by transcript matrix.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for fragmenting double-stranded cDNA on a support, comprising:
(a) contacting double-stranded input cDNA with a plurality of transposomes that are tethered to a support, wherein the tethered transposomes comprise:
(i) a transposase and
(ii) a nucleic acid that is tethered to the support and comprises a support barcode and a PCR primer amplification sequence,
wherein the contacting step (a) results in tagmentation of the cDNA to produce first cDNA fragments that are tethered to the support; and
(b) contacting the product of step (a) with a plurality of untethered transposomes, wherein the untethered transposomes comprise:
(i) a transposase and
(ii) a nucleic acid that comprises a PCR primer amplification sequence, wherein the contacting step (b) results in tagmentation of at least some of the first cDNA fragments of (a) to produce second cDNA fragments that have: i) the support barcode and ii) a PCR primer amplification sequence at both ends.

2. The method of claim 1, wherein the nucleic acid of (a)(ii) comprises i. transposon ends that are bound to the transposase, ii. the barcode, iii the PCR primer amplification sequence and iv. an end that is tethered to the support.

3. The method of any prior claim, wherein the nucleic acid of (b)(ii) comprises i. transposon ends that are bound to the transposase and ii. the PCR primer amplification sequence.

4. The method of any prior claim, wherein the method comprises amplifying the first and second fragments to produce amplification products.

5. The method of claim 4, further comprising sequencing the amplification products to produce sequence reads.

6. The method of claim 5, further comprising grouping the sequence reads using the support barcode.

7. The method of claim 5, further comprising linking the sequence reads that have the same barcode to reconstitute a full-length cDNA.

8. The method of any prior claim, wherein the support is a bead.

9. The method of any prior claim, wherein the support barcode is a bead-specific barcode.

10. The method of any prior claim, wherein the double-stranded input cDNA comprises a PCR primer amplification sequence at one or both ends.

11. The method of any prior claim, wherein the double-stranded cDNA comprises a molecular index at one or both ends.

12. The method of claim 11, wherein the molecular index is a cell type-specific barcode.

13. The method of claim 11, wherein the molecular index is a diseased cell type-specific barcode.

14. The method of any one of claims 1-13, wherein the double-stranded input cDNA has a length of greater than about 500 bases; and wherein the second cDNA fragments have lengths of no more than 700 nucleotides.

15. The method of claim 14, wherein each of the second cDNA fragments has a length of from about 100 nucleotides to about 700 nucleotides.

16. The method of claim 14 or claim 15, wherein the double-stranded input cDNA has a length of from about 500 bases to about 20,000 bases.

17. The method of any one of claims 1-16, wherein the input cDNA comprises a nucleotide sequence that is complementary to a target RNA isoform.

18. The method of claim 17, wherein the target RNA isoform is indicative of a disease state of a cell.


SEQUENCE LISTING (4 sequences)

SEQ ID NO: 1 - DNA, 29 bp, Unknown
  sequence binding site
aatgatacggcgaccaccgagatctacac

SEQ ID NO: 2 - DNA, 24 bp, Unknown
  sequence binding site
caagcagaagacggcatacgagat

SEQ ID NO: 3 - DNA, 19 bp, Unknown
  transposase recognition site
agatgtgtataagagacag

SEQ ID NO: 4 - DNA, 19 bp, Unknown
  transposase recognition site
ctgtctcttatacacatct